(19) United States
(12) Patent Application Publication
(10) Pub. No.: US 2004/0186318 A1
(43) Pub. Date: Sep. 23, 2004

(54) Method for producing (2e)-2-(hydroxyphenyl-2-(alkoxyimino)-n-methylacetamides

(76) Inventors: Holger Weintritt, Langenfeld (DE); Reinhard Lantzsch, Wuppertal (DE); Thorsten Muh, Leverkusen (DE)

Correspondence Address: BAYER CROPSCIENCE LP, Patent Department, 100 BAYER ROAD, PITTSBURGH, PA, 15205-9741, US

(21) Appl. No.: 10/485,065

(22) PCT Filed: Jul. 19, 2002
(86) PCT No.: PCT/EP02/08040

(30) Foreign Application Priority Data
Aug. 1, 2001 (DE) 101 37 728.2

(51) Int. Cl. C07C 249/12

(52) U.S. Cl. 564/134

(57) ABSTRACT

The invention relates to a novel process for preparing (2E)-2-(hydroxyphenyl)-2-(alkoxyimino)-N-methylacetamides.

The invention relates to a novel process for preparing (2E)-2-(hydroxyphenyl)-2-(alkoxyimino)-N-methylacetamides.

It is already known that 2-(2-(hydroxyphenyl)-2-(methoxyimino)-N-methylacetamide (C) is obtained as a mixture of E- and Z-isomers when methyl 2-(2-hydroxyphenyl)-2-(methoxyimino)acetate (A) or 1-benzofuran-2,3-dione-3-(O-methyloxime) (B) is reacted with methylamine (compare, for example, EP-A 398 692 or WO 95/24396):

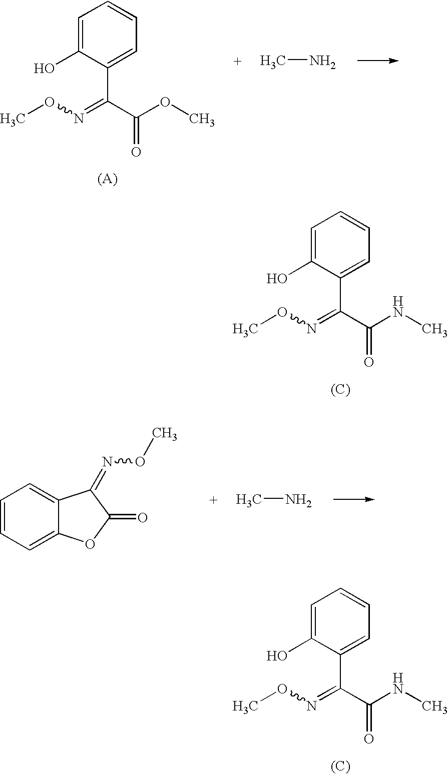

The disadvantages of these processes are firstly that the starting materials are difficult to obtain and, secondly, that, using this process, 2-(2-hydroxyphenyl)-2-(methoxyimino)-N-methylacetamide (C) is obtained as a mixture of E- and Z-isomers.

It has now been found that (2E)-2-(hydroxyphenyl)-2-(alkoxyimino)-N-methylacetamides of the general formula (I),

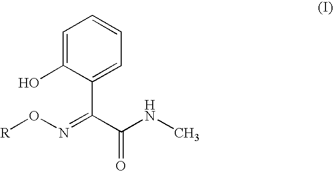

in which

R represents C₁-C₆-alkyl which is unsubstituted or mono- to trisubstituted by fluorine

are obtained when

a) the compound of the formula (II)

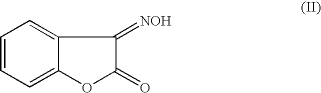

is reacted with methylamine—or an acid addition complex thereof,

if appropriate in the presence of a diluent and if appropriate in the presence of an acid acceptor, and

b) the resulting compound of the formula (III)

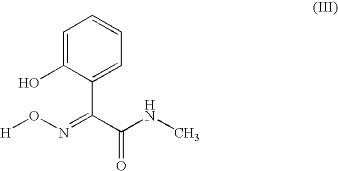

is, if appropriate without work-up,

reacted with an alkylating agent of the formula (IV)

R—X (IV)

in which

R is as defined above and

X represents chlorine, bromine, iodine, —O—CO—O—R, —O—SO₂—R, or —O—SO₂—O—R, where R is as defined above,

if appropriate in the presence of a diluent and if appropriate in the presence of an acid acceptor, if appropriate in the presence of a phase-transfer catalyst.

In the definition of R, the saturated C₁-C₆-alkyl hydrocarbon chains are in each case straight-chain or branched. C₁-C₆-alkyl represents in particular methyl, ethyl, n- or i-propyl, n-, s- or i-butyl, n-pentyl or i-pentyl.

R represents in particular trifluoromethyl, difluoromethyl, fluoromethyl, methyl, ethyl or n-propyl.

R preferably represents methyl, ethyl or n-propyl.

R particularly preferably represents methyl.

The general or preferred radical definitions listed above apply both to the end products of the formula (I) and, correspondingly, to the starting materials or intermediates required in each case for the preparation.

The specific radical definitions given for these radicals in the respective combinations or preferred combinations of radicals are, independently of the particular combination of radicals given, also replaced by any radical definitions of other preferred ranges.

It is extremely surprising that in the process according to the invention in process step a) the ring opening of the compound of the formula (II) takes place selectively with formation of the E isomers of the 2-(2-hydroxyphenyl)-2-(alkoxyimino)-N-methylacetamides, since ring opening of 1-benzofuran-2,3-dione-3-(O-methyloxime) (B) with methylamine affords a mixture of E- and Z-isomers (compare, for example, EP-A 398 692 or WO 95/24396). It is also surprising that in the alkylation of the compound of the formula (III) the alkylation occurs selectively on the oxime oxygen.

Process a) according to the invention has a number of advantages. Thus, the E-isomers of the 2-(2-hydroxyphenyl)-2-(alkoxyimino)-N-methylacetamides can be prepared selectively in high yields and purities from readily available starting materials under conditions without any technical problems.

The compound of the formula (II) is already known and can be prepared by known methods (compare, for example, DE-A-2162717, EP-A-711 751).

The methylamine—or its acid addition complexes—furthermore required as starting material for carrying out process step a) of the process according to the invention is a commercially available chemical synthesis. It can be employed either as gas or as an alcoholic or aqueous solution. Preferred acid addition complexes are the hydrochlorides, hydrogensulphates or sulphates.

Suitable alkylating agents of the formula (IV) furthermore required as starting materials for carrying out process step b) of the process according to the invention are all chemical compounds capable of transferring alkyl groups. Preference is given to chloromethane, bromomethane, iodomethane, dimethyl sulphate and dimethyl carbonate. Particular preference is given to dimethyl sulphate and chloromethane.

Suitable diluents for carrying out the process a) according to the invention are all inert organic solvents. These include, by way of example and by way of preference, aliphatic, alicyclic or aromatic hydrocarbons, such as, for example petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as, for example, acetonitrile, propionitrile, n- or i-butyronitrile or benzonitrile; amides, such as N,N-dimethylformamide, N,N-dimethylacetamide, N-methylformanilide, N-methylpyrrolidone or hexamethyl-phosphoric triamide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

Process step a) of the process according to the invention is, if appropriate, carried out in the presence of a suitable acid acceptor. Suitable acid acceptors are all customary inorganic or organic bases. These include, by way of example and by way of preference, alkaline earth metal or alkali metal hydroxides, alkoxides, acetates, carbonates or bicarbonates, such as, for example, sodium methoxide, sodium ethoxide, potassium tert-butoxide, sodium hydroxide, potassium hydroxide, sodium acetate, potassium acetate, sodium carbonate, potassium carbonate, potassium bicarbonate or sodium bicarbonate, and also tertiary amines, such as for example, trimethylamine, triethylamine, tributylamine, N,N-dimethylaniline, N,N-dimethyl-benzylamine, pyridine, N-methylpiperidine, N-methylmorpholine, N,N-dimethyl-aminopyridine, diazabicyclooctane (DABCO), diazabicyclononene (DBN) or diazabicycloundecene (DBU). In process a), preference is given to using potassium carbonate, sodium hydroxide or potassium hydroxide.

When carrying out process step a) of the process according to the invention, the reaction temperatures can be varied within a relatively wide range. In general, the process step is carried out at temperatures of from 15° C. to reflux temperatures, preferably at temperatures of from 20° C. to 40° C., in particular at temperatures of from 20° C. to 25° C.

Process step a) of the process according to the invention is generally carried out under atmospheric pressure. However, it is also possible to operate under elevated or reduced pressure—in general between 0.1 bar and 10 bar.

For carrying out process step a) of the process according to the invention for preparing compounds of the formula (I), in general from 1 to 4 mol, preferably from 1 to 2 mol, of methylamine are employed per mole of the compound of the formula

Process step a) of the process according to the invention for preparing compounds of the formula (I) is generally carried out as follows: methylamine is added to the compound of the formula (II), preferably in the presence of a diluent. The mixture is stirred, if appropriate at elevated temperature, until the reaction has gone to completion. After the reaction has ended, the mixture is worked up in a customary manner.

Suitable diluents for carrying out process step b) of the process according to the invention are all inert organic solvents. These include, by way of example and by way of preference, alphatic, alicyclic or aromatic hydrocarbons, such as, for example petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane, 1,2-diethoxyethane or anisole; ketones, such as, for example, acetone, butanone, methyl isobutyl ketone or cyclohexanone nitriles, such as, for example, acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

Process step b) of the process according to the invention is, if appropriate, carried out in the presence of a suitable acid acceptor. Suitable acid acceptors are all customary inorganic or organic bases. These include, by way of example and by way of preference, alkaline earth metal or alkali metal hydroxides, alkoxides, acetates, carbonates or bicarbonates, such as, for example, sodium methoxide, sodium ethoxide, potassium tert-butoxide, sodium hydroxide, potassium hydroxide, sodium acetate, potassium acetate, sodium carbonate, potassium carbonate, potassium bicarbonate or sodium bicarbonate, and also tertiary amines, such as, for example, trimethylamine, triethylamine, tributylamine, N,N-dimethylaniline, N,N-dimethyl-benzylamine, pyridine, N-methylpiperidine, N-methylmorpholine, N,N-dimethyl-aminopyridine, diazabicyclooctane (DABCO), diazabicyclononene (DBN) or diazabicycloundecene (DBU). In process b), preference is given to using potassium carbonate, sodium hydroxide or potassium hydroxide.

Process step b) of the process according to the invention is, if appropriate, carried out in the presence of a suitable phase-transfer catalyst. These include, by way of example and by way of preference, quaternary ammonium salts, such as, for example, tetrabutylammonium bromide, chloride, hydrogensulphate or sulphate, methyl trioctylammonium bromide or chloride, hydrogensulphate or sulphate or 4-dimethylaminio-N-(2-ethylhexyl)pyridinium bromide or chloride, hydrogensulphate or sulphate, quartemary phosphonium salts, such as, for example, tributyltetradecylphosphonium bromide or chloride, tetraphenylphosphonium bromide or chloride, crown ethers, such as, for example, dibenzo-18-crown-6, guanidinium salts, such as, for example, hexaalkylguanidinium chloride, and also polyethylene glycol derivatives. In process step b), preference is given to using tetrabutylammonium bromide or tetrabutylammonium chloride.

When carrying out process step b) of the process according to the invention, the reaction temperatures can be varied within a relatively wide range. In general, the process step is carried out at temperatures of from −20° C. to 80° C., preferably at temperatures of from 0° C. to 60° C.

Process step b) of the process according to the invention is generally carried out under atmospheric pressure. However, it is also possible to operate under elevated or reduced pressure—in general between 0.1 bar and 10 bar.

For carrying out process step b) of the process according to the invention for preparing the compounds of the formula (I), in general from 1 to 15 mol, preferably from 1 to 4 mol, of alkylating agent of the formula (IV) is employed per mole of the compound of the formula (III).

Process step b) of the process according to the invention is generally carried out as follows: the compound of the formula (III) is, preferably in the presence of a diluent, mixed with a base and, if appropriate, a phase-transfer catalyst. The alkylating agent of the formula (IV) is added and the mixture is stirred, if appropriate at elevated temperature, until the reaction has gone to completion. After the reaction has ended, the mixture is worked up in a customary manner.

The process according to the invention is used in particular for preparing (2E)-2-(hydroxyphenyl)-2-(methoxyimino)-N-methylethanamide, an important intermediate for the preparation of pesticides (EP-A-937 050). Using the process according to the invention, (2E)-2-(hydroxyphenyl)-2-(methoxyimino)-N-methylethanamide is obtained in constantly high purities and good yields. Accordingly, the novel process facilitates the preparation of known pesticides.

The examples below serve to illustrate the invention. However, the invention is not limited to the examples.

### PREPARATION EXAMPLES

### Example 1

(2E)-2-(hydroxyimino)-2-(2-hydroxyphenyl)-N-methylethanamide:

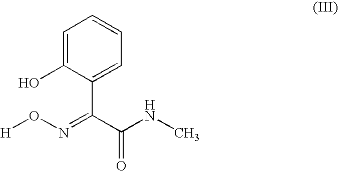

Process step a)

5.0 g of benzofuran-2,3-dione 3-oxime are dissolved in 20 ml of methanol, and, at room temperature, 3.4 g of methylamine (40% strength in methanol) are added over a period of 15 minutes. The mixture is then stirred at room temperature for 2 hours and concentrated to dryness. This gives (2E)-2-(hydroxyimino)-2-(2-hydroxyphenyl)-N-methylethanamide (GC: 98.8%) in quantitative yield.

(2E)-2-(hydroxyphenyl)-2-(methoxyimino)-N-methylethanamide:

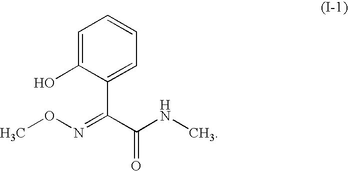

Process step b)

5.0 g of (2E)-2-(hydroxyimino)-2-(2-hydroxyphenyl)-N-methylethanamide and 7.1 g of potash are initially charged in 45 ml of acetone, and a solution of 3.6 g of dimethyl sulphate and 25 ml of acetone is added at room temperature over a period of 4 hours. The mixture is stirred at room temperature for another 1.5 hours, the solvent is distilled off, 150 ml of water are added, the pH is adjusted to 6-7 using hydrochloric acid and the mixture is extracted three times with methylene chloride. The combined organic extracts are washed with water, dried with sodium sulphate and then concentrated to dryness. This gives 4.7 g of (2E)-2-(hydroxyphenyl)-2-(methoxyimino)-N-methylethanamide (HPLC: 83.6%; yield: 73.3% of theory).

1. Process for preparing compounds of the general formula (I), 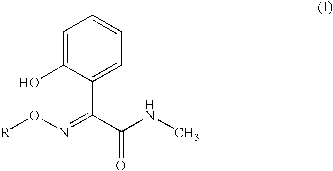
in which
R represents C₁-C₆-alkyl which is unsubstituted or mono- to trisubstituted by fluorine,
characterized in that
a) the compound of the formula (II) 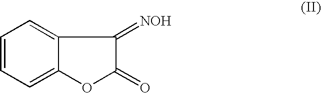
is reacted with methylamine—or an acid addition complex thereof—,
if appropriate in the presence of a diluent and if appropriate in the presence of an acid acceptor,
and
b) the resulting compound of the formula (III) 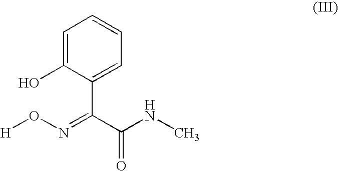
is, if appropriate without work-up,
reacted with an alkylating agent of the formula (IV)
R—X (IV)
in which
R is as defined above and
X represents chlorine, bromine, iodine, —O—CO—O—R, —O—SO₂—R, or —O—SO₂—O—R, where R is as defined above,
if appropriate in the presence of a diluent and if appropriate in the presence of an acid acceptor, if appropriate in the presence of a phase-transfer catalyst.

2. Process according to claim 1, characterized in that process steps a) and b) are carried out as a one-pot process.

3. Process according to claim 1 or 2, characterized in that the alkylating agent of the formula (IV) used in process step b) is dimethyl sulphate or methyl chloride.

4. Process according to at least one of claims 1 to 3, characterized in that process step a) is carried out at temperatures of from 20° C. to reflux temperature.

5. Process according to at least one of claims 1 to 4, characterized in that process step b) is carried out at temperatures from −20° C. to 80° C.

6. Process according to at least one of claims 1 to 5, characterized in that, for carrying out process step a) for preparing the compounds of the formula (I), from 1 to 4 mol of methylamine are employed per mole of the compound of the formula (II).

7. Process according to at least one of claims 1 to 6, characterized in that, for carrying out process step b) for preparing the compounds of the formula (I), from 1 to 15 mol of alkylating agent of the formula (IV) are employed per mole of the compound of the formula (III).

8. Process according to at least one of claims 1 to 7, characterized in that R represents methyl.
